(19) United States
(12) Patent Application Publication
(10) Pub. No.: US 2024/0404650 A1
(43) Pub. Date: Dec. 5, 2024

(54) GENERATIVE MACHINE LEARNING MODEL ASSISTED STATISTICAL INVERSION FOR BOREHOLE SENSING

(71) Applicant: Halliburton Energy Services, Inc., Houston, TX (US)

(72) Inventors: Xusong WANG, Singapore (SG); Christopher Michael JONES, Houston, TX (US); Xiang WU, Singapore (SG); Jichun SUN, Singapore (SG)

(73) Assignee: Halliburton Energy Services, Inc., Houston, TX (US)

(21) Appl. No.: 18/535,058

(22) Filed: Dec. 11, 2023

Related U.S. Application Data
(60) Provisional application No. 63/469,931, filed on May 31, 2023.

(51) Int. Cl. G16C 20/70; G01V 20/00

(52) U.S. Cl. CPC G16C 20/70; G01V 20/00

(57) ABSTRACT

Aspects of the subject technology relate to systems, methods, and computer readable media for applying generative machine learning for performing statistical inversion in borehole sensing. A method can comprise implementing an inversion workflow for borehole sensing. The method can also comprise applying a generative machine learning network technique to sample candidate material variables as part of the inversion workflow.

### CROSS-REFERENCE TO RELATED APPLICATIONS

This application claims benefit of U.S. Provisional Application No. 63/469,931 filed May 31, 2023, which is incorporated herein by reference.

### TECHNICAL FIELD

The present technology pertains to applying generative machine learning for performing statistical inversion in borehole sensing and, more particularly, to applying a generative machine learning network technique to sample candidate material variables as part of the inversion workflow.

### BACKGROUND

In current borehole well logging process, such as acoustic, electromagnetic and nuclear logging, inversion is used in identifying properties from logged measurements. Machine learning is an Artificial Intelligence (AI) technology that maps inputs to outputs. Machine learning can be used to generate a forward modelling numerical solution. It is difficult, however, to utilize machine learning in the inversion process. While mappings from material properties to physical measurements/tool responses is stable, mapping from measurements/tool responses to a material property model can be unstable.

### BRIEF DESCRIPTION OF THE DRAWINGS

In order to describe the manner in which the features and advantages of this disclosure can be obtained, a more particular description is provided with reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings.

FIG. 1A is a schematic diagram of an example logging-while-drilling (LWD) wellbore operating environment, in accordance with various aspects of the subject technology;

FIG. 1B is a schematic diagram of an example downhole environment having tubulars, in accordance with various aspects of the subject technology.

FIG. 2 illustrates a conventional inversion workflow.

FIG. 3 illustrates an example inversion workflow using machine learning, in accordance with various aspects of the subject technology.

FIGS. 4-5 illustrate example generative models for modeling material properties and measurements, in accordance with various aspects of the subject technology.

FIG. 6 illustrates a neural network (NN) for mapping between hidden layers, in accordance with various aspects of the subject technology.

FIG. 7 depicts the information flow while training a NN for use in inversion in borehole sensing, in accordance with various aspects of the subject technology.

FIG. 8 illustrates the structure and information flow in a NN while in use for forward modeling in borehole sensing, in accordance with various aspects of the subject technology.

FIG. 9 is a flowchart of an example process of training a NN, in accordance with various aspects of the subject technology.

FIG. 10 illustrates the structure and information flow in a NN while in use for inversion in borehole sensing, in accordance with various aspects of the subject technology.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description, or can be learned by practice of the principles disclosed herein. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

As discussed previously, current borehole well logging processes, such as acoustic, electromagnetic and nuclear logging, use inversion to identify properties of the formation from logged measurements. Machine learning is an AI technology that maps inputs to outputs. Machine learning can be used to generate a forward modelling numerical solution. It is difficult, however, to utilize machine learning in the inversion process. While mappings from material properties to physical measurements/tool responses is stable, mapping from measurements/tool responses to material property model can be unstable.

The technology described herein relates to utilizing generative machine learning models in the inversion process. The benefits of using machine learning in inversion can include the algorithm efficiency. Current bottlenecks for well logging measurement are in the running time. Run time processes can be sped up using generative machine learning models in the inversion process.

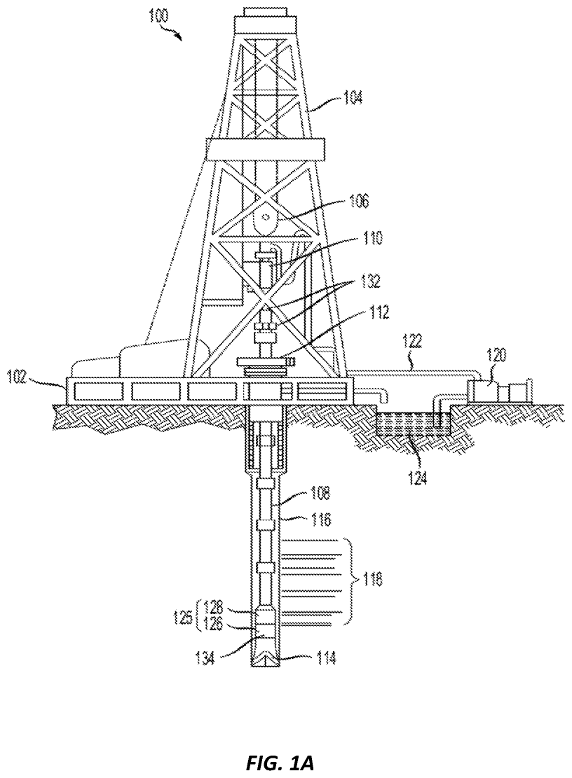

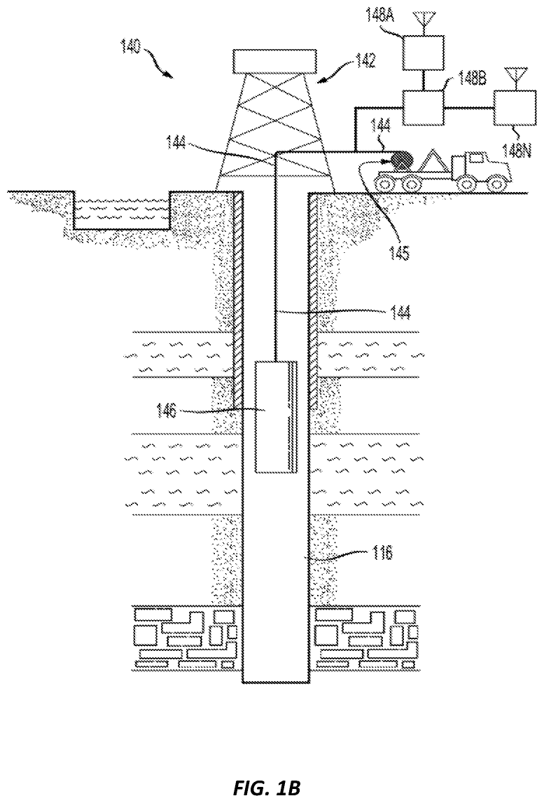

FIG. 1A is a schematic diagram of an example LWD wellbore operating environment 100, in accordance with various aspects of the subject technology. LWD typically incorporates sensors that acquire formation data. Specifically, the drilling arrangement shown in FIG. 1A can be used to gather formation data through an electromagnetic imaging tool as part of logging the wellbore. The drilling arrangement of FIG. 1A also exemplifies what is referred to as measurement-while-drilling (MWD) which utilizes sensors to acquire data from which the wellbore's path and position in three-dimensional space can be determined. FIG. 1A shows a drilling platform 102 equipped with a derrick 104 that supports a hoist 106 for raising and lowering a drill string 108. The hoist 106 suspends a top drive 110 suitable for rotating and lowering the drill string 108 through a well head 112. A drill bit 114 can be connected to the lower end of the drill string 108. As the drill bit 114 rotates, it creates a wellbore 116 that passes through various subterranean formations 118. A pump 120 circulates drilling fluid through a supply pipe 122 to top drive 110, down through the interior of drill string 108 and out orifices in drill bit 114 into the wellbore. The drilling fluid returns to the surface via the annulus around drill string 108, and into a retention pit 124. The drilling fluid transports cuttings from the wellbore 116 into the retention pit 124 and the drilling fluid's presence in the annulus aids in maintaining the integrity of the wellbore 116. Various materials can be used for drilling fluid, including oil-based fluids and water-based fluids.

In certain embodiments, logging tools 126 are integrated into the bottom-hole assembly 125 near the drill bit 114. During operations such as using the drill bit 114 to extend the wellbore 116 through the formations 118 and removing the drill string 108 from the wellbore 116, logging tools 126 collect measurements relating to various formation properties as well as the orientation of the tool and various other drilling conditions. In certain embodiments, the logging tool 126 includes tools for collecting measurements in a drilling scenario, such as the electromagnetic imaging tools described herein. In certain embodiments, each of the logging tools 126 includes one or more tool components spaced apart from each other and communicatively coupled by one or more wires and/or other communication arrangement. In certain embodiments, the logging tools 126 includes one or more computing devices communicatively coupled with one or more of the tool components. In certain embodiments, the one or more computing devices are configured to control or monitor a performance of the tool, process logging data, and/or carry out one or more aspects of the methods and processes of the present disclosure.

In certain embodiments, the bottom-hole assembly 125 includes a telemetry sub 128 to transfer measurement data to a surface receiver 132 and to receive commands from the surface. In at least some cases, the telemetry sub 128 communicates with a surface receiver 132 by wireless signal transmission, e.g., using mud pulse telemetry, EM telemetry, or acoustic telemetry. In certain embodiments, one or more of the logging tools 126 communicate with a surface receiver 132 by a wire, such as wired drill pipe. In some instances, the telemetry sub 128 does not communicate with the surface, but rather stores logging data for later retrieval at the surface when the logging assembly is recovered. In certain embodiments, one or more of the logging tools 126 receive electrical power from a wire that extends to the surface, including wires extending through a wired drill pipe. In certain embodiments, power is provided from one or more batteries or via power generated downhole.

Collar 134 is a frequent component of a drill string 108 and generally resembles a very thick-walled cylindrical pipe, typically with threaded ends and a hollow core for the conveyance of drilling fluid. Multiple collars 134 can be included in the drill string 108 and are constructed and intended to be heavy to apply weight on the drill bit 114 to assist the drilling process. Because of the thickness of the collar's wall, pocket-type cutouts or other type recesses can be provided into the collar's wall without negatively impacting the integrity (strength, rigidity and the like) of the collar as a component of the drill string 108.

FIG. 1B is a schematic diagram of an example downhole environment having tubulars, in accordance with various aspects of the subject technology. An example system 140 is depicted for conducting downhole measurements after at least a portion of a wellbore 116 has been drilled and the drill string removed from the well. An electromagnetic imaging tool can be operated in the example system 140 to log the wellbore. A downhole tool is shown having a tool body 146 in order to carry out logging and/or other operations. For example, instead of using the drill string 108 of FIG. 1A to lower the downhole tool, which can contain sensors and/or other instrumentation for detecting and logging nearby characteristics and conditions of the wellbore 116 and surrounding formations, a wireline conveyance 144 can be used. The tool body 146 can be lowered into the wellbore 116 by wireline conveyance 144. The wireline conveyance 144 can be anchored in the drill rig 142 or by a portable means such as a truck 145. The wireline conveyance 144 can include one or more wires, slicklines, cables, and/or the like, as well as tubular conveyances such as coiled tubing, joint tubing, or other tubulars. The downhole tool can include an applicable tool for collecting measurements in a drilling scenario, such as the electromagnetic imaging tools described herein.

In certain embodiments, the illustrated wireline conveyance 144 provides power and support for the tool, as well as enabling communication between data processors 148A-N on the surface. In certain embodiments, the wireline conveyance 144 includes electrical and/or fiber optic cabling for carrying out communications. The wireline conveyance 144 is sufficiently strong and flexible to tether the tool body 146 through the wellbore 116, while also permitting communication through the wireline conveyance 144 to one or more of the processors 148A-N, which can include local and/or remote processors. The processors 148A-N can be integrated as part of an applicable computing system, such as the computing device architectures described herein. In certain embodiments, power is supplied via the wireline conveyance 144 to meet power requirements of the tool. In certain embodiments, e.g., slickline or coiled tubing configurations, power is supplied by a battery or a downhole generator.

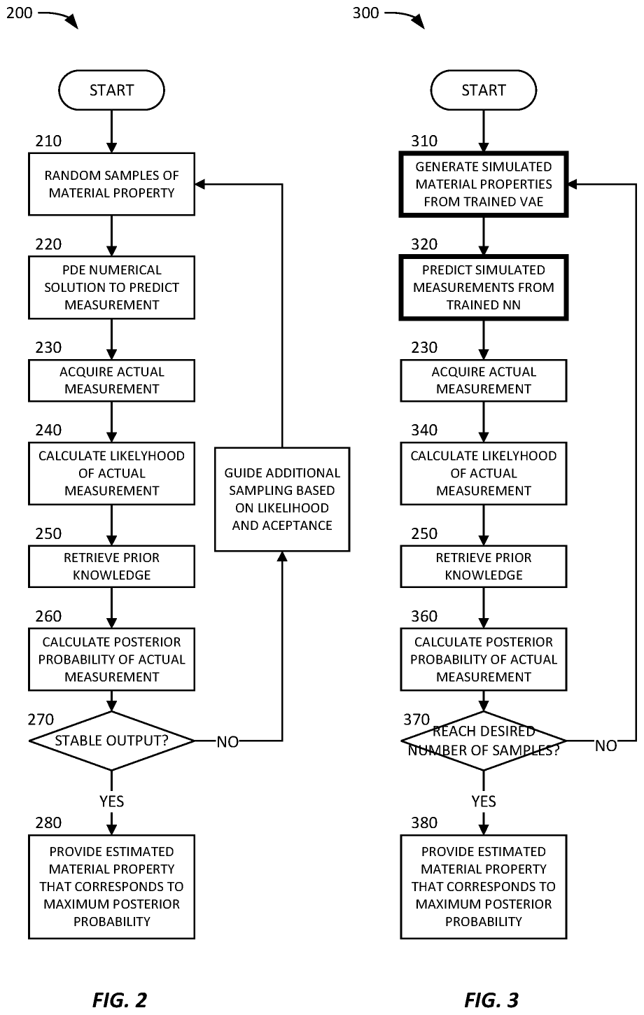

FIG. 2 illustrates a traditional statistical inversion workflow 200. Statistical inference consists in learning about what we do not observe based on what we observe. In borehole sensing, we can observe measurements made by logging tools but we cannot observe the actual formation material. There is prior knowledge about the material properties of formation materials. Applying a statistical view, the following parameters are defined:
p(m) prior knowledge of distribution of material properties
p(d|m) “likelihood” probability of measurement d for a given m
p(m|d) “posterior” probability of material property m for a given d

In these parameters, m is the material property model, d is the measurement (physical response) data, and p is the probability function. Note that p(d|m) needs forward modelling to map the material property model m to the respective physical response data d.

Step 210 samples p(m) to generate a distribution and step 220 applies a numerical model to determine the likelihood distribution p(d|m), e.g., a numerical simulation incorporating partial differential equations (PDEs). Forward modeling can be computationally expensive, especially if the simulation process is time-consuming or requires many iterations to produce accurate results.

Step 230 acquires a actual measurement, e.g., data from the logging tool, and step 240 calculates the probability of obtaining the value of the actual measurement of step 230 given the likelihood from step 220, i.e., the posterior probability p(m|d), which is given by Eqn. 1. In certain embodiments, the material property model m that corresponds to maximum posterior probability is the inverted material property model.

$$ρ ( m \vert d ) = \frac{ρ ( m ) \times ρ ( d \vert m )}{\int ρ ( d \vert m ) \times ρ ( m ) dm} \qquad Equation 1$$

Although the integral in the denominator can be computed without too much difficulty in simple cases, it can become intractable in higher dimensions and exact computation of the posterior probability p(m|d) is practically infeasible. An approximation technique must be used to get a solution. Conventional approaches to overcome the difficulties in this method include use of a Markov Chain Monte Carlo (MCMC) method.

A system with an input m and an output d can be treated as a process with a time-sequence of output values. The output of a Gaussian process is unrelated to the output at any prior time. In a Markov process, the distribution of a future state is dependent only upon the present state and not on any prior state (memoryless). A sequence of Markov outputs with discrete times is referred to as a “Markov Chain.”

MCMC algorithms are aimed at generating samples from a given probability distribution, including complex, high-dimension probability distributions. In order to produce samples, as done in step 210, a Markov Chain is defined with a stationary distribution, e.g., the prior knowledge. Then, we can simulate a random sequence of states from that Markov Chain that is long enough to be sufficiently close to the steady state and then keep some generated states as our samples. MCMC can be still be computationally expensive, which limits its usefulness in real-time analysis of borehole logging measurements.

FIG. 3 illustrates an example inversion workflow 300 using machine learning, in accordance with various aspects of the subject technology. In this example, steps 210, 220 of workflow 200 are replaced with equivalent steps 310, 320 that use machine learning to develop a Neural Network (NN) that provides the distribution of measurements derived from a model of the material properties. Step 310 uses a generative NN to provide random samples of the material properties m and step 320 uses a NN to quickly and accurately map the m values to measurement values d.

The improvement of workflow 300 over workflow 200 is the increase in speed of the overall process due to the elimination of computationally intensive (slow) simulation processes in step 220. This enables the borehole operators to see the nature of the formation while drilling and make adjustments to the drilling process, thereby producing a better wellbore.

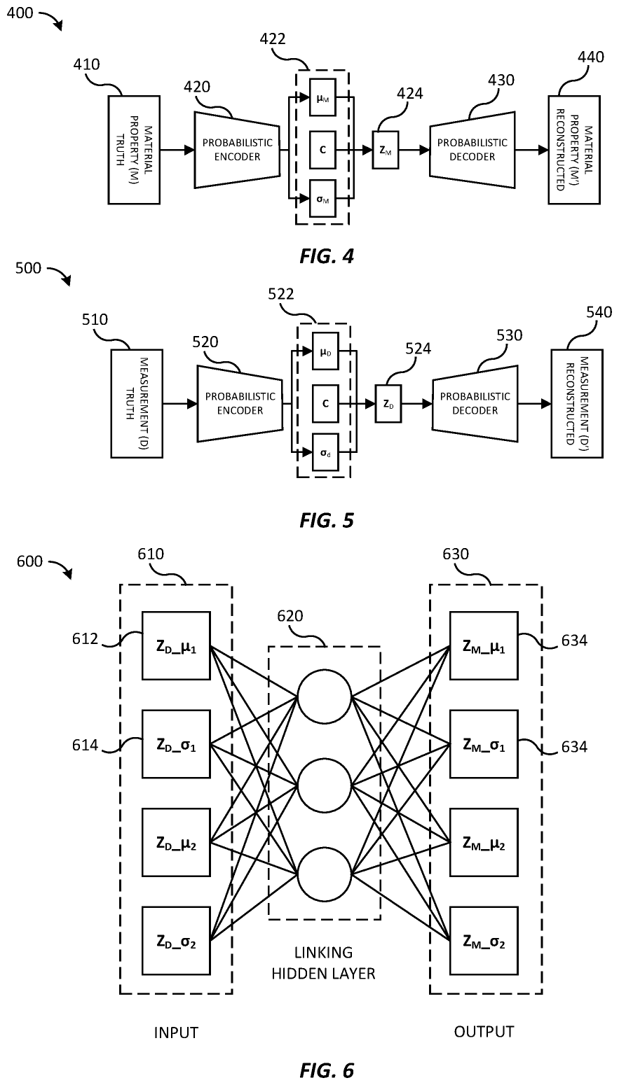

FIG. 4 illustrates an example generative model 400 for modeling material properties, in accordance with various aspects of the subject technology. Generative models model the joint probability distribution of the input features (X) and target labels (Y), denoted as P (X,Y). The mapping is X|→P (X,Y) and includes 1-to-many mappings. Such models aim to learn how the data is generated and attempt to capture the underlying structure or process that produces the data. By learning the joint distribution, generative models can generate new samples that resemble the training data in terms of statistical characteristic. In certain embodiments, the generative model is one of a Naive Bayes classifier, a Gaussian Mixture Model (GMM), a Hidden Markov Model (HMM), a Variational Autoencoder (VAE), a Generative Adversarial Network (GAN), and a Restricted Boltzmann Machine (RBM).

The example generative model 400 is a VAE that comprises an input state 410 that feeds a probabilistic encoder 420 that generates a hidden layer Zₘ 424, which in this example is regularized in block 422 to have a mean μₘ and a variance Om and a constant Cₘ. The model 400 also has a probabilistic decoder 430 that transforms the hidden layer Zₘ 424 to reconstructed values M′ in the original space as output 440. Training VAE 400 uses training data sets of a range of values of M and compares the resultant M′ values to the corresponding input M using a cost/error function, as is known to those of skill in the art.

FIG. 5 illustrates an example VAE 500 for modeling measurements, in accordance with various aspects of the subject technology. The input state 510 that feeds a probabilistic encoder 520 that generates a hidden layer Z_{d} 524, which in this example is regularized in block 522 to have a mean μ_{d} and a variance Od and a constant C_{d}. The model 400 also has a probabilistic decoder 530 that transforms the hidden layer Z_{d} 524 to reconstructed values d′ in the original space as output 540.

FIG. 6 illustrates a NN for mapping between hidden layers, in accordance with various aspects of the subject technology. Discriminative models describe the conditional probability of the target labels given the input features, denoted as X|→Y. Discriminative models include 1-to-1 mapping and bijective or surjective mapping and focus on finding the decision boundary that separates different classes, or predicts the target labels directly, without attempting to model the data generation process. Example discriminative models include Logistic Regression, Random Forests, Support Vector Machines (SVM), and Gradient Boosting Machines (GBM). In certain embodiments, discriminative models is a deep learning neural network, e.g., an Artificial Neural Network (ANN), a Convolutional Neural Network (CNN), and a Recursive Neural Network (RNN).

In certain embodiments, the NN 600 is configured with an input 610 that corresponds to the hidden layer Z_{d} of VAE 500, comprising pairs of the regularization mean e.g., Zd_μ₁ 612, and variance, e.g., Zd_θ₁ 614. Similarly, the output 630 corresponds to the hidden layer Zₘ of VAE 400 and includes regularization mean e.g., Zₘ_μ₁ 612, and variance, e.g., Zₘ_θ₁ 614.

The NN 600 comprises at least one layer of a linking hidden layer 620 connected between the components of the input 610 and the output 630. In certain embodiments, the NN 600 is a discriminative model. In certain embodiments, the NN 600 is an ANN.

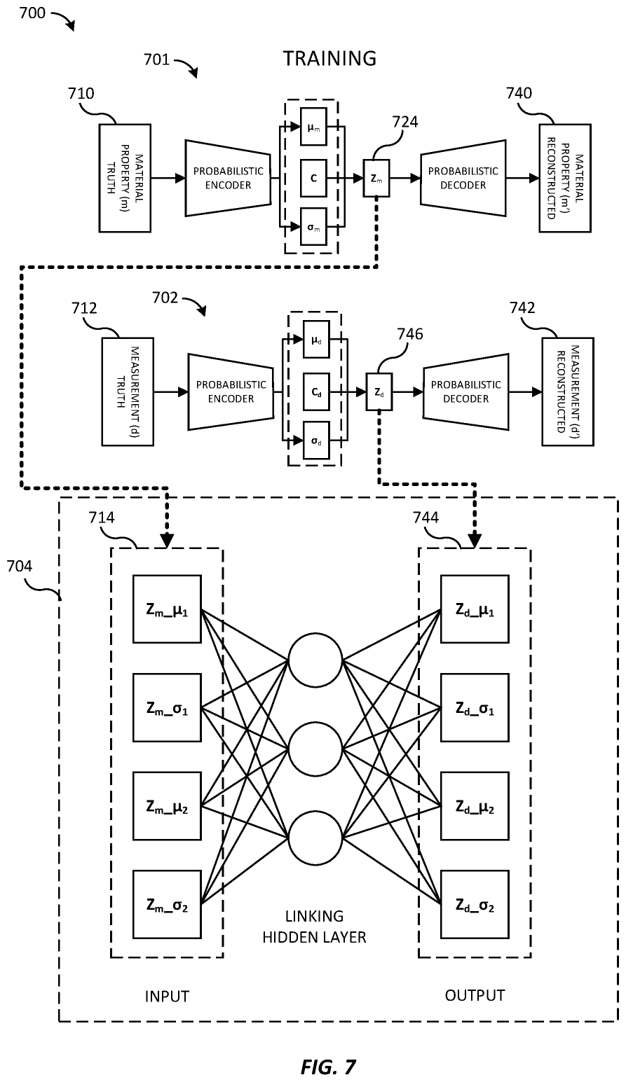

FIG. 7 depicts an arrangement 700 for training a NN for use in inversion in borehole sensing, in accordance with various aspects of the subject technology. A first data set 710, 740 is used to train a generative model, e.g., a VAE 701, to develop the hidden layer 724. A second data set 712, 742 is used to train a generative model, e.g., a VAE 702, to develop the hidden layer 726. After training of the VAEs 701, 702 is completed, the hidden layer 724 is replicated as the input 714 of a discriminative model, e.g., an ANN 704, and the hidden layer 726 is replicated as the output 744 and the ANN 704 trained using the parameters of the input 714 and output 744.

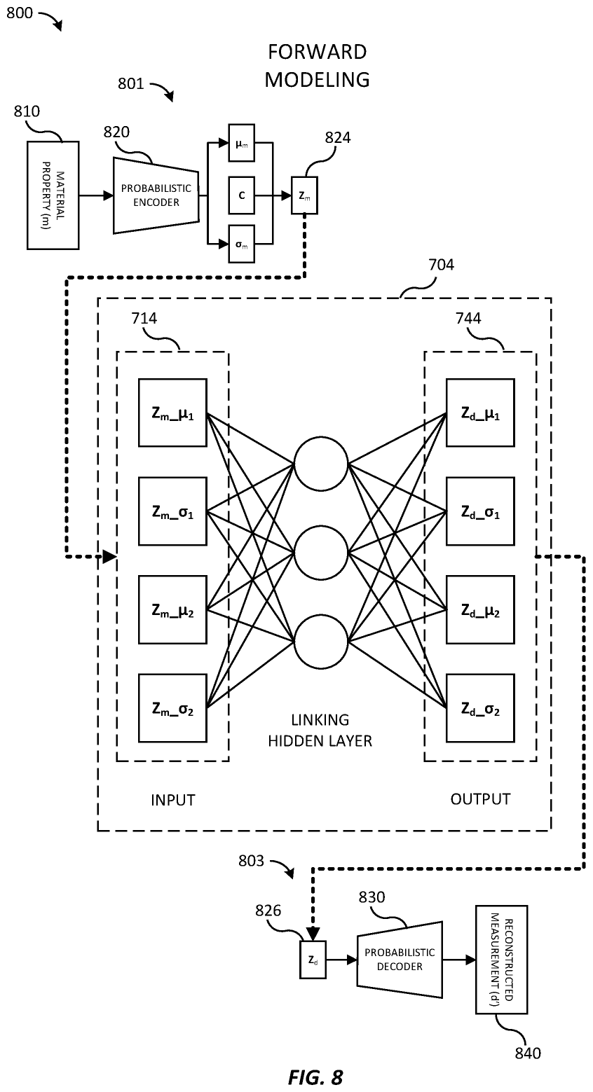

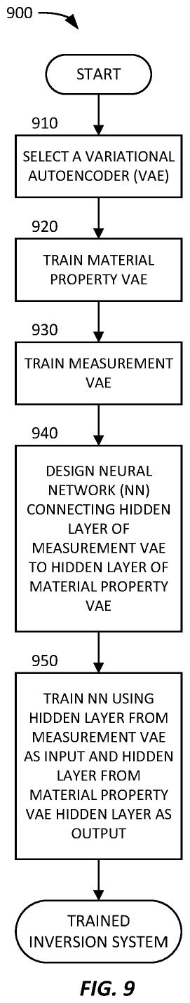

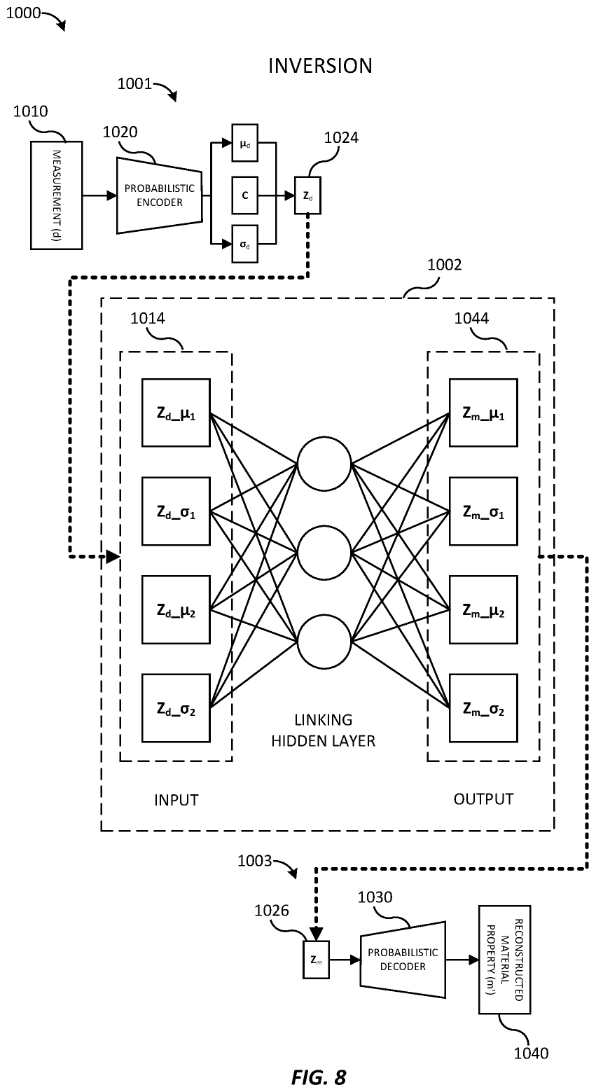

FIG. 8 illustrates the structure and information flow in a ML system 800 while in use for forward modeling in borehole sensing, in accordance with various aspects of the subject technology. The system 800 comprises an encoder portion 801 of the VAE 701 of FIG. 7, the discriminative model 704 of FIG. 7, and a decoder portion 803 of the VAE 702 of FIG. 7. In this configuration, the output 744 is replicated as the hidden layer 826 of the portion 803. In use, for example the flowchart 300 of FIG. 3, this system implements steps 310, 320 to provide predicted values of the measurements.

FIG. 9 is a flowchart of an example process 900 of training a system, e.g., system 800, for use in inversion processing of wellbore measurements, in accordance with various aspects of the subject technology. In step 910, an appropriate generative model is selected for the measurement model, e.g., VAE 500 and for the material property model, e.g., VAE 400. In certain embodiments, a different model is selected for the models 400, 500. Selection of a model includes selection of the type and parameters associated with that particular model, as known to those of skill in the art. The material property VAE is trained in step 920 and the measurement model is trained in step 930, as known to those of skill in the art.

Step 940 designs the inversion system NN, e.g., system 800, by connecting the hidden layer of the measurement model to the input of a discriminative model, e.g., ANN 704, and the output of the discriminative model to the hidden layer of the material property model. Step 950 trains the NN using the hidden layers of the trained material property and measurement models from steps 920, 930. Completion of step 950 provides a trained inversion system, e.g., system 800 of FIG. 8.

FIG. 10 illustrates the structure and information flow in a ML system 1000 while in use for inversion in borehole sensing, in accordance with various aspects of the subject technology. The system 1000 comprises an encoder portion 1001 of the VAE 702 of FIG. 7, a discriminative model 1002 that is a reversed-image of model 704 of FIG. 7, and a decoder portion 1003 of the VAE 701 of FIG. 7. In this configuration, the hidden layer 1024 is replicated as input 1014 and the output 1044 is replicated as the hidden layer 1026 of the portion 1003. Training of this system will generally follow the methods described with reference to FIG. 7 modified to reflect the configuration of FIG. 10.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can include hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the disclosed concepts may be otherwise variously embodied and employed, and that the appended claims are intended to be construed to include such variations, except as limited by the prior art. Various features and aspects of the above-described subject matter may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the method, algorithms, and/or operations described above. The computer-readable data storage medium may form part of a computer program product, which may include packaging materials.

The computer-readable medium may include memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

In the above description, terms such as “upper,” “upward,” “lower,” “downward,” “above,” “below,” “downhole,” “uphole,” “longitudinal,” “lateral,” and the like, as used herein, shall mean in relation to the bottom or furthest extent of the surrounding wellbore even though the wellbore or portions of it may be deviated or horizontal. Correspondingly, the transverse, axial, lateral, longitudinal, radial, etc., orientations shall mean orientations relative to the orientation of the wellbore or tool. Additionally, the illustrate embodiments are illustrated such that the orientation is such that the right-hand side is downhole compared to the left-hand side.

The term “coupled” is defined as connected, whether directly or indirectly through intervening components, and is not necessarily limited to physical connections. The connection can be such that the objects are permanently connected or releasably connected. The term “outside” refers to a region that is beyond the outermost confines of a physical object. The term “inside” indicates that at least a portion of a region is partially contained within a boundary formed by the object. The term “substantially” is defined to be essentially conforming to the particular dimension, shape or another word that substantially modifies, such that the component need not be exact. For example, substantially cylindrical means that the object resembles a cylinder, but can have one or more deviations from a true cylinder.

Although a variety of information was used to explain aspects within the scope of the appended claims, no limitation of the claims should be implied based on particular features or arrangements, as one of ordinary skill would be able to derive a wide variety of implementations. Further and although some subject matter may have been described in language specific to structural features and/or method steps, it is to be understood that the subject matter defined in the appended claims is not necessarily limited to these described features or acts. Such functionality can be distributed differently or performed in components other than those identified herein. The described features and steps are disclosed as possible components of systems and methods within the scope of the appended claims.

Claim language reciting “an item” or similar language indicates and includes one or more than one of the items. For example, claim language reciting “a part” means one part or multiple parts. Moreover, claim language reciting “at least one of” a set indicates that one member of the set or multiple members of the set satisfy the claim. For example, claim language reciting “at least one of A and B” means A, B, or A and B.

What is claimed is:

1. A method of mapping a first parameter to a second parameter, the method comprising:
training a first machine learning (ML) model comprising a first hidden layer to accurately return a reconstructed first parameter as a first output when provided with the first parameter as a first input, thereby producing a trained first hidden layer;
training a second ML model comprising a second hidden layer to accurately return a reconstructed second parameter as a second output when provided with the second parameter as a second input, thereby producing a trained second hidden layer;
training a third ML model comprising a linking hidden layer to accurately return the trained second hidden layer as a third output when provided with the trained first hidden layer as a third input, thereby producing a trained linking hidden layer;
constructing a ML system comprising a first portion of the first ML model, a second portion of the second ML model, and the third ML model, with the first hidden layer connected to the third input and the third output connected to the second hidden layer, such that providing a first plurality of values of the first parameter as a system input produces a second plurality of values of the second parameter as a system output.

2. The method of claim 1, wherein the first ML model and the second ML model both comprise a generative model.

3. The method of claim 2, wherein the generative model comprises a variational autoencoder (VAE).

4. The method of claim 1, wherein the third ML model comprises a discriminative model.

5. The method of claim 4, wherein the discriminative model comprises an artificial neural network (ANN).

6. The method of claim 1, wherein the first parameter comprises a material property and the second parameter comprises a measurement.

7. The method of claim 6, wherein the first parameter is unobservable and the second parameter is observable.

8. The method of claim 1, wherein the first parameter comprises a measurement and the second parameter comprises a material property.

9. The method of claim 8, wherein the first parameter is observable and the second parameter is unobservable.

10. A system comprising:
a processor configured to accept a signal; and
a computer-readable memory coupled to the processor, the memory comprising a machine learning (ML) system that comprises:
a first machine learning (ML) model comprising a first input, a first trained hidden layer, and a first output;
a second ML model comprising a second input, a second trained hidden layer, and a second output; and
a third ML model comprising a third input coupled to the first hidden layer, a trained linking hidden layer, and a third output coupled to the second hidden layer;
wherein:
the first ML model is trained to return a reconstructed first parameter as the first output when provided with a true first parameter as the first input, thereby producing the trained first hidden layer;
the second ML model is trained to accurately return a reconstructed second parameter as the second output when provided with a true second parameter as the second input, thereby producing the trained second hidden layer;
the third ML model is trained to accurately return the trained second hidden layer as the third output when provided with the trained first hidden layer as the third input, thereby producing the trained linking hidden layer; and
providing a first plurality of values of the first parameter as a system input produces a second plurality of values of the second parameter as a system output.

11. The system of claim 10, wherein the first ML model and the second ML model both comprise a generative model.

12. The system of claim 11, wherein the generative model comprises a variational autoencoder (VAE).

13. The system of claim 10, wherein the third ML model comprises a discriminative model.

14. The system of claim 13, wherein the discriminative model comprises an artificial neural network (ANN).

15. The system of claim 10, wherein the first parameter comprises a material property and the second parameter comprises a measurement.

16. The system of claim 15, wherein the first parameter is unobservable and the second parameter is observable.

17. The system of claim 10, wherein the first parameter comprises a measurement and the second parameter comprises a material property.

18. The system of claim 17, wherein the first parameter is observable and the second parameter is unobservable.

19. A computer-readable memory comprising a machine learning (ML) system that comprises:
a first machine learning (ML) model comprising a first input, a first trained hidden layer, and a first output;
a second ML model comprising a second input, a second trained hidden layer, and a second output; and
a third ML model comprising a third input coupled to the first hidden layer, a trained linking hidden layer, and a third output coupled to the second hidden layer;
wherein:
the first ML model is trained to return a reconstructed first parameter as the first output when provided with a true first parameter as the first input, thereby producing the trained first hidden layer;
the second ML model is trained to accurately return a reconstructed second parameter as the second output when provided with a true second parameter as the second input, thereby producing the trained second hidden layer;
the third ML model is trained to accurately return the trained second hidden layer as the third output when provided with the trained first hidden layer as the third input, thereby producing the trained linking hidden layer; and
providing a first plurality of values of the first parameter as a system input produces a second plurality of values of the second parameter as a system output.

20. The memory of claim 19, wherein the first ML model and the second ML model both comprise a generative model and the third ML model comprises a discriminative model.
